SEQUENCE LISTING (2 sequences)

SEQ ID NO: 1 - protein, 252 aa, Hepatitis C virus
MRGSTLHGPTPLLYRLGAVQNEVTLTHPITKYIMTCMSADLEVVTSTWVLVGGVLAALAAYCLSTGCVVIVGRIVLSGKPAVIPDREVLYREFDEMEECSQHLPYIEQGMALAEQFKQKALGLLQTASRQAEVIAPAVQTNWQKLEAFWAKHMWNFISGIQYLAGLSTLPGNPAIASLMAFTAAVTSPLTTSQTLLFNILGGWVAAQLAAPGAATAFVGAGLAGAAIGSVGLGKVLVDILAGYGAGVAGALV

SEQ ID NO: 2 - protein, 42 aa, Hepatitis C virus
IIPDREVLYREFDEMEECSQHLPYIEQGMMLAEQFKQKALGL

(19) United States
(12) Patent Application Publication
(10) Pub. No.: US 2006/0035335 A1
(43) Pub. Date: Feb. 16, 2006

(54) Use of HCV proteins

(76) Inventors: Kingston Henry Gordon Mills, Dublin (IE); Miriam Therse Brady, Maynooth (IE)

Correspondence Address: JACOBSON HOLMAN PLLC, 400 SEVENTH STREET N.W., SUITE 600, WASHINGTON, DC, 20004, US

(21) Appl. No.: 11/245,153

(22) Filed: Oct. 7, 2005

Related U.S. Application Data
(63) Continuation of application No. PCT/IE04/00054, filed on Apr. 8, 2004.

(30) Foreign Application Priority Data
Apr. 11, 2003 (IE) 2003/0279

(51) Int. Cl. C12Q 1/68; C12P 21/06

(52) U.S. Cl. 435/69.1; 435/6

(57) ABSTRACT

A Hepatitis C virus (HCV) protein such as a non-structured protein 4 (NS4) or a non-structured protein 3 (NS3) or a derivative or fragment or variant or peptide thereof or product of cells activated by the agent is useful in the treatment and/or prophylaxis of an inflammatory and/or an immune-mediated disorder. The agent can also be used as a vaccine adjuvant.

The invention relates to the use of Hepatitis C virus (HCV) proteins or derivative thereof.

### INTRODUCTION

Hepatitis C virus (HCV) is a single-stranded, positive sense RNA species responsible for the majority of blood-borne non-A, non-B hepatitis and now affects approximately 2% of the world's population (1). Approximately 80% of HCV-infected patients develop chronic infection, with about 20% of these eventually developing severe complications, including liver cirrhosis or heptaocellular carcinoma (2). It has been suggested that clearance of HCV infection is dependent on vigorous multispecific immune responses, particularly the secretion of type 1 cytokines, to both structural and non-structural proteins by both CD4⁺ Th1 cells and CD8⁺ cytotoxic T lymphocytes (CTL) (3-6). However, in chronically HCV infected individuals, including those that develop liver disease, the virus persists in the face of HCV-specific antibodies and cellular immune responses (3, 7, 8). The development of chronicity has been linked to weak or absent Th1 responses and the presence of Th2 cytokines (9, 10), suggesting that HCV may encode proteins that facilitate evasion of immune surveillance, or that induce an inappropriate response for viral clearance. However disease progression and hepatic injury has also been linked to high serum IL-12 levels and active Th1-type responses or reduced IL-10 in the liver (11-13).

Viruses that persistently infect the host have developed multiple strategies to evade or subvert immune responses, including interference with antigen presentation, and the production of cytokine or chemokine homologs that circumvent the inflammatory response (14, 15). In particular, the cytokine IL-10 has been exploited by pathogens, including HIV (16, 17), rhinovirus (18), murine gammaherpesvirus-68 (19), Bordetella pertussis (20) and mycobacteria (21), to suppress cellular immune responses and delay or prevent their elimination from the host. Studies with IL-10-defective mice and anti-IL-10 antibodies have provided further evidence of a role for IL-10 in the regulation of protective immunity to a number of chronic diseases, including visceral leishmaniasis (22) filariasis (23), schistosomiasis (24), leprosy (25) and tuberculosis (26). IL-10 has also been implicated in viral persistence in chronically HCV infected individuals (9, 27). It has been reported that in patients with persistent HCV infection, spontaneous IL-10 production is greater (28), and serum IL-10 levels are enhanced (29, 30), which has also been implicated in recurrence of hepatitis C after liver transplantation (31). Furthermore, IL-10 polymorphisms were more frequent in HCV infected patients with virologically sustained response to antiviral therapy than in nonresponders (32). Evidence is also emerging that T cells, which secrete IL-10 and/or TGF-β, termed regulatory T cells (Tr cells), are induced during HCV infection (30). These cells function to maintain immunological tolerance, but are also capable of suppressing pathogen-specific immune responses and facilitating the development of chronic infections (33). HCV core-specific type 1 Tr (Tr1) clones established from peripheral blood of individuals chronically infected HCV have been shown to secrete IL-10 and IL-5, but not IL-4 or IFN-γ (30).

There is a need to develop therapeutic agents for use in the treatment and/or prophylaxis of an inflammatory and/or immune-mediated disorder and/or disorders associated with transplantations.

### STATEMENTS OF INVENTION

According to the invention there is provided a therapeutic composition comprising a Hepatitis C virus (HCV) agent comprising a HCV protein or derivative or mutant or fragment or variant or peptide thereof which suppresses inflammatory cytokine production and/or promotes IL-10 production in vitro.

The invention also provides a vaccine adjuvant comprising a Hepatitis C virus (HCV) agent comprising a HCV protein or derivative or mutant or fragment or variant or peptide thereof or product of cells activated by the agent.

The invention also provides the use of an agent comprising a Hepatitis C virus (HCV) protein or derivative or mutant or fragment or variant or peptide or product of cells activated by the agent for the treatment and/or prophylaxis of an inflammatory and/or immune-mediated disorder and/or disorders associated with transplantation.

According to the invention there is also provided a method for the treatment and/or prophylaxis of an inflammatory and/or immune-mediated disorder and/or disorders associated with transplantation comprising the step of administering an agent comprising a Hepatitis C virus (HCV) protein or derivative or mutant or fragment or variant or peptide thereof.

In one embodiment the HCV protein is non-structural protein 4 (NS4) or a derivative or mutant or fragment or variant or peptide thereof.

In another embodiment the HCV protein is non-structural protein 3 (NS3) or a derivative or mutant or fragment or variant or peptide thereof.

In one case the agent suppresses inflammatory cytokine production. The agent also promotes IL-10 production, particularly by peripheral blood mononuclear cells (PBMC) and/or monocytes.

In one case the agent or product thereof inhibits dendritic cell activation. In one case the agent or product thereof may also inhibit the induction or activation of Th1 or Th2 cells.

In one case the agent or product thereof modulates toll-like receptor ligand-induced NFκB activation.

In one case the agent modulates inflammatory cytokine production induced by infection or trauma.

The disorder may be a sepsis or acute inflammation induced by infection, trauma or injury.

The disorder may be a chronic inflammatory disease, graft rejection or graft versus host disease.

The disorder may be an immune mediated disease involving Th1 responses.

In one embodiment the agent is used for the prophylaxis and/or treatment of a NFκB related disease or condition.

The disorder may be an immune mediated disease involving inflammatory cytokines, including TNF-α and IL-1.

The disorder may be any one or more of Crohn's disease, inflammatory bowel disease, multiple sclerosis, type 1 diabetes, rheumatoid arthritis, systemic lupus erythematosus, uveitis, allergy or asthma.

The invention also provides a method of inhibiting Toll-like receptor (TLR) dependant signalling comprising administration of an effective amount of Hepatitis C virus (HCV) protein or a derivative, mutant, variant, fragment or peptide thereof.

In another embodiment the invention provides a method for the treatment of infectious disease or cancer comprising the step of administering an agent comprising a Hepatitis C virus (HCV) protein or derivative or mutant or fragment or variant or peptide thereof.

The invention also provides a method for the treatment of and/or prophylaxis of asthma and/or allergy comprising the step of administering an agent comprising a Hepatitis C virus (HCV) protein or derivative or mutant or fragment or variant or peptide thereof.

The agent may be in a form for oral, intranasal, intravenous, intradermal, subcutaneous or intramuscular administration.

In another aspect the invention provides the use of an agent comprising a Hepatitis C virus (HCV) protein or derivative or mutant or variant or peptide or product of cells activated by the agent for the prophylaxis and/or treatment of diseases or conditions involving Toll-like receptor (TLR) dependant signalling.

The invention further provides the use of an agent comprising a Hepatitis C virus (HCV) protein or derivative or mutant or fragment or variant or peptide or product of cells activated by the agent for the prophylaxis and/or treatment of asthma or allergy.

### BRIEF DESCRIPTION OF THE INVENTION

The invention will be more clearly understood from the following description of two embodiments thereof, given by way of example only with reference to the accompanying drawings in which:

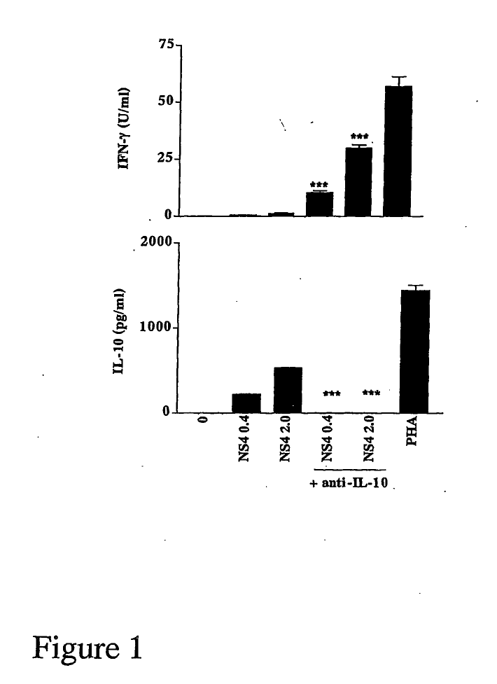

FIG. 1 are bar charts illustrating that anti-IL-10 restores defective antigen-specific IFN-γ production by PBMC from HCV-infected patients. PBMC (2×10⁶/ml) from HCV antibody positive, PCR positive patients were stimulated with rNS4 (0.4 and 2.0 μg/ml), PHA or medium only for 72 h, in the presence or absence of a neutralizing IL-10 antibody (10 μg/ml). Results are mean ±SE of cytokine concentrations for triplicate culture and are representative of nine patients. ***P<0.001 cells stimulated with NS4 alone versus NS4 with anti-IL-10;

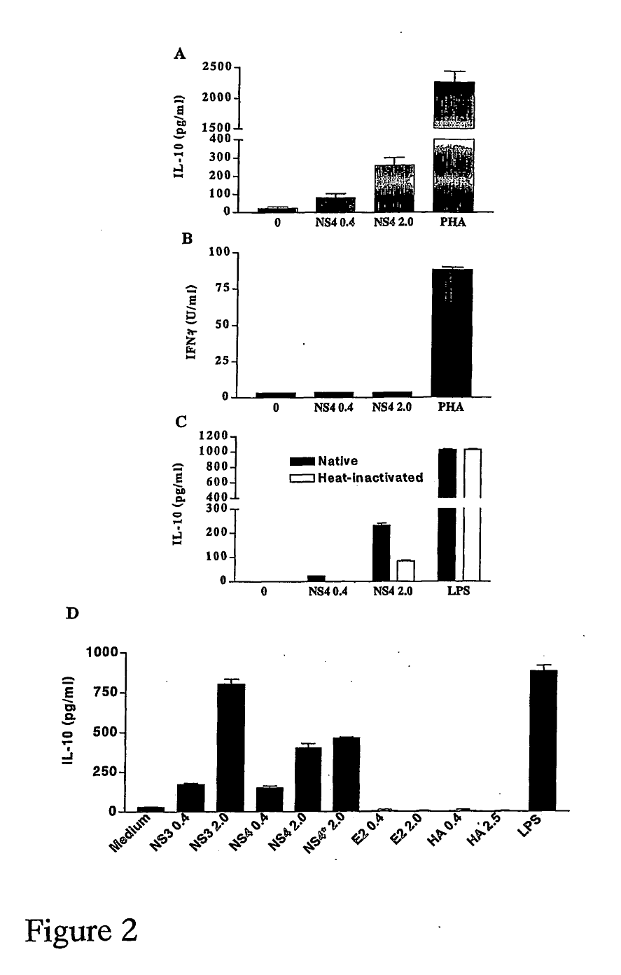

FIG. 2 are bar charts illustrating that NS4 stimulates IL-10 production (A), but not IFN-γ production (B) by PBMC from normal subjects. PBMC (1×10⁶/ml) from normal donors, were stimulated with rNS4 (0.4 and 2.0 μg/ml), medium only, or PHA as a positive control and IL-10 and IFN-γ concentrations in the supernatants were assessed after 24 h. Cytokine production was also assessed in response to heat inactivated NS4 (C). LPS was used as a positive control. Results express the means (±SE) cytokine concentrations for triplicate cultures and are representative of 24 donors. HCV NS4 and NS3, but not E2, stimulate IL-10 production from normal PBMC (D). PBMC (1×10⁶/ml) from normal donors, were stimulated with rNS4 (0.4 and 2.0 mg/ml), rNS3 (0.4 and 2.0 mg/ml) (Mikrogen antigens), NS4* (2.0 μg/ml) and HCV E2 (0.4 and 2.0 mg/ml) (Austral antigens), influenza virus HA (0.4 and 2.5 mg/ml) or with LPS (1 mg/ml). IL-10 concentrations in the supernatants were assessed after 24 hr. Results are mean (±SE) cytokine concentrations for triplicate cultures and are representative of 3 experiments.

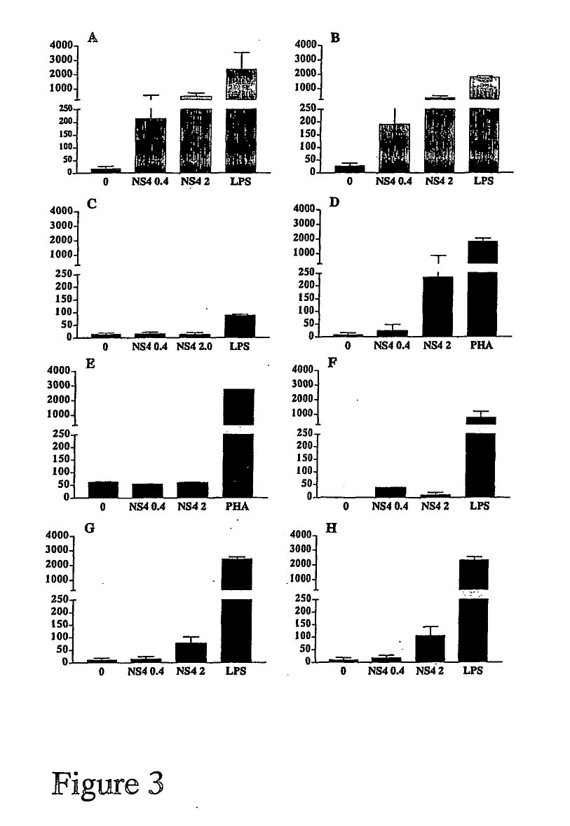

FIG. 3 are bar charts illustrating that monocytes are the source of innate IL-10 produced in response to rNS4. PBMC (A), E⁻ (13), E⁺ cells (C), adherent cells (D), non-adherent cells (E) iDC F, CD14⁺ monocytes (G) and CD11b⁺ monocytes (H) (1×10⁶/ml) from normal individuals, were stimulated with rNS4 (0.4 and 2.0 μg/ml). LPS (1 μg/ml) or PHA (20 μg/ml) were used as positive controls. Results express the means (±SE) IL-10 concentrations for triplicate cultures and re representative of four experiments;

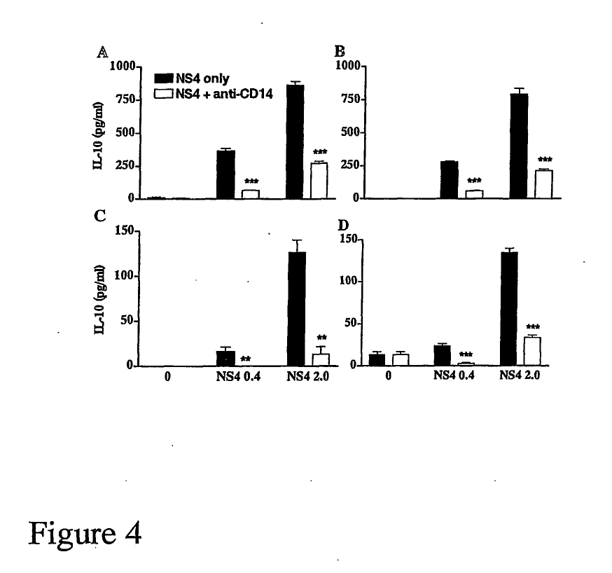

FIG. 4 are bar charts illustrating that IL-10 production by NS4-stimulated monocytes is mediated by CD14. PBMC (A), E⁻ cells (B), CD14⁺ monocytes (C) and CD11b⁺ monocytes (D) (1×10⁶/ml), were stimulated with rNS4 (0.4 and 2.0 μg/ml) in the presence or absence of anti-CD14 (10 μg/ml) Results are mean (±SE) IL-10 concentrations for triplicate cultures and are representative of four experiments. **P<0.01, ***P<0.001 Cells stimulated with NS4 alone versus NS4 with anti-CD14;

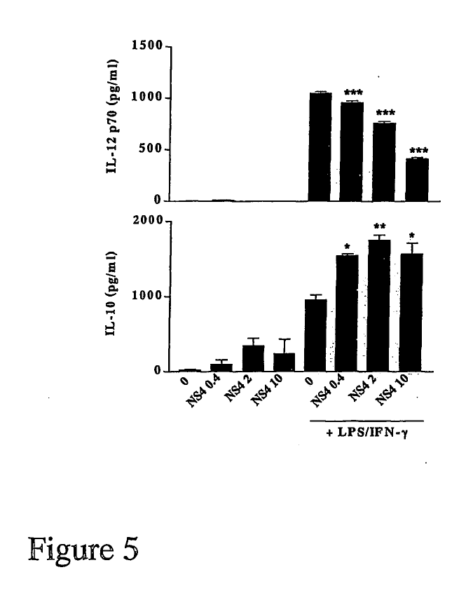

FIG. 5 are bar charts illustrating that IL-12 production by monocytes is inhibited by NS4. PBMC (1×10⁶/ml) were stimulated for 24 h with LPS (1 μg/ml) and IFN-γ (20 ng/ml), rNS4 (0.4, 2.0 and 10 μg/ml), or with LPS and IFN-γ following a 2 h pre-incubation with rNS4. Stimulation with medium only was used as negative control. Results are mean (±SE) cytokine concentrations for triplicate cultures, and are representative of four experiments. *P<0.05 **P<0.01, ***P<0.001 versus LPS and IFN-γ stimulation alone;

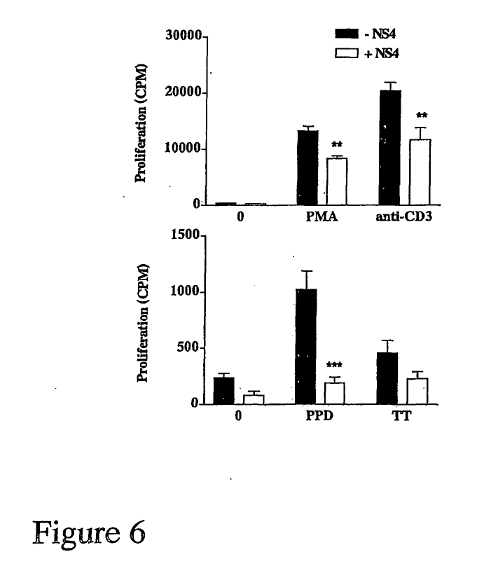

FIG. 6 are bar charts illustrating that NS4 inhibits antigen-specific T-cell proliferation to polyclonal activators and recall antigens. PBMCs (1×10⁶/ml) were stimulated with anti-CD3 (10 μg/ml), PMA (0.2 μg/ml) (A), PPD (500 U/ml) or TT (5 Lf/ml) (B), in the presence or absence of rNS4 (2 μg/ml). T-cell proliferation was measured on day 3 (for anti-CD3, PMA stimulation) and day 5 (for PPD, TT stimulation) by measurement of ³H thymidine incorporation for the last 18 h of culture. Results are mean cpm (±SE) for triplicate cultures. **P<0.01, ***P<0.001 cells cultured with NS4 versus without NS4;

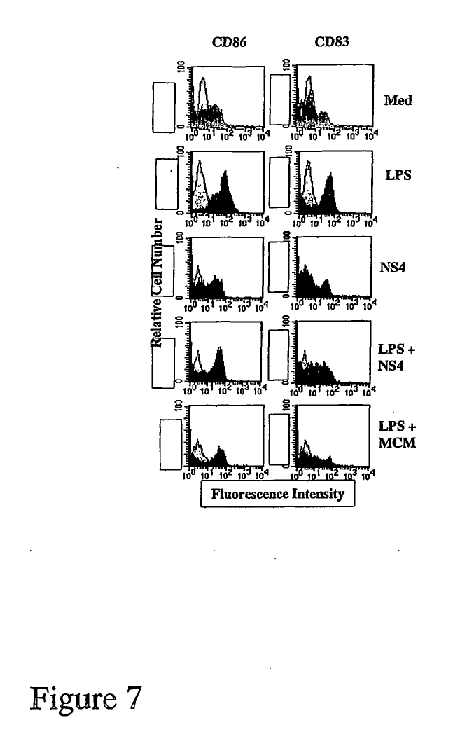

FIG. 7 are FACS analysis showing NS4-stimulated monocyte products modulate DC maturation. Blood monocyte-derived DC were stimulated with LPS (1 μg/ml), NS4 (2 μg/ml). NS4-monocyte conditioned medium (NS4MCM; 10%), LPS and NS4 or LPS and MCM. After 24 h of culture, cells were washed and immunofluorescence analysis performed for CD86 and CD83 (black histograms), or isotype-matched control antibodies (grey histograms).

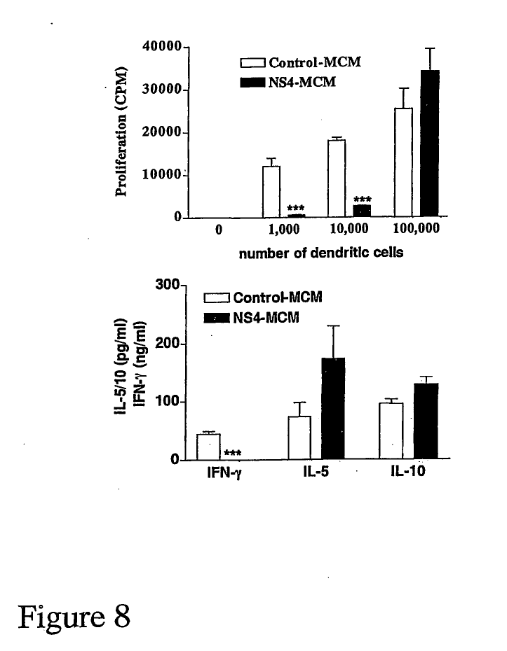

FIG. 8 are bar charts illustrating that products of NS4-stimulated monocytes inhibit T cell allostimulatory activity of DC. NS4MCM and control-MCM was prepared by stimulating purified monocytes with rNS4 or medium only respectively and supernatants removed after 24 hr. DCs were incubated with NS4-MCM or control-MCM for 2 hr, and after washing, DC (1,000-100,000) were used to simulate purified allogeneic T cells (1×10⁶/ml). (A) Proliferation was determined after 5 days by ³H thymidine incorporation. (B) Supernatants were removed after 72 hr of culture, and concentrations of IFN-γ, IL-5 and IL-10 were assessed by immunoassay. Results represent mean CPM (±SE) for triplicate cultures. Results for cytokine analysis represent T cell responses with a single concentration of DC (10⁴/ml for IFN-γ and 10⁵/ml for IL-5 and IL-10). **P<0.01, ***P<0.001 NS4-MCM versus control MCM.

### DETAILED DESCRIPTION

We have found that proteins from HCV can induce the, production of an anti-inflammatory cytokine and inhibit inflammatory responses. Proteins from HCV, in particular HCV non-structural protein 4 (NS4) and NS3, were found to suppress cellular immunity by inducing IL-10 and inhibiting IL-12 production by cells of the innate immune system, which in turn drive the activation of dendritic cells (DC) that drive the differentiation of Th1 cells. HCV NS4 was shown to inhibit innate and adaptive immune response.

NS4 stimulated CD14dependant induction of IL-10 from monocytes, the products of which inhibited dendritic cells (DC) maturation and priming of Th1 responses in vitro. Furthermore, defective NS4specific IFN-γ production in chronically HCV infected individuals was restored by co-incubation with anti-IL-10 antibodies. The encoding of a multifunctional protein capable of directly stimulating an immunosuppressive and anti-inflammatory cytokine indicates a previously unrecognised strategy by HCV to subvert protective immunity or a strategy by the host to limit inmmunopathology in the liver.

Viral infection elicits a wide spectrum of host immune responses, involving both innate and adaptive defence mechanisms and these responses are usually capable of clearing the virus in immunocompetent individuals. However, a number of viruses, including pox viruses, HIV, hepatitis B virus and HCV have evolved strategies that enable them to evade or subvert host immune responses involved in viral clearance and persist indefinitely in a high proportion of infected individuals (14-19).

It was found in the present invention that persistence of HCV in chronically infected individuals was in part facilitated by the induction of regulatory or anti-inflammatory cytokines that inhibit putative protective cellular immune responses.

A number of theories have been put forward to explain persistence of HCV despite the induction of potent HCV-specific immune responses in chronically infected individuals. The high rate of genetic variations during viral replication results in the generation of mutants that escape immune recognition by T cells and antibody (15). Another possibility is that the virus infects cells of the immune system itself, which represent a privileged site that cannot be reached by virus-specific T-cell responses. Other immune subversion mechanisms include viral inhibition of antigen processing or presentation (14), modulation of the response to cytotoxic mediators, or immunological tolerance to HCV antigens. HCV may also encode proteins that facilitate evasion of immune surveillance, or that induce an inappropriate response for viral clearance. Several HCV proteins have been shown to interfere with cell signalling in host cells. NS5A suppresses the catalytic activity of IFN-induced double stranded RNA-activated protein kinase (PKR), an important component of cellular anti-viral response, allowing HCV to escape anti-viral effects of IFN (35). Furthermore, NS5A activates NF-κB and STAT-3 through activation of protein tyrosine kinase (PTK) promoting cell survival with a possible role in progression to hepatocelluar carcinoma (36). The HCV core protein induces expression of SOCS3 and inhibits IFN-α induced tyrosine phosphorylation and activation of STAT-1 (37).

The term HCV protein as used in this specification includes at least 10 mature proteins encoded by the viral RNA core, envelope glycoproteins (E1, E2, p7) and non structural proteins (NS2, NS3, NS4A, NS4B, NS5A and NS5B). The invention also includes a mutant or fragment or derivative or variant or peptide of any of these as well as products of cells activated by the proteins.

Thus, the invention relates to the use of a HCV agent comprising a HCV protein as a therapeutic or a vaccine adjuvant. The agent is not limited to a HCV protein per se but also includes a derivative or fragment or variant thereof or peptide or product of cells activated by the agent. For example, we describe below that a 42 amino acid fragment of NS4 (corresponding to amino acids 1694-1735 with an N-terminal super oxidase dismutase label) retained the immunomodulatory activity observed with the NS4-NS3 construct (corresponding to amino acids 1616-1862), demonstrating that a fragment or peptide of NS4 could be used in place of the full-length protein. Furthermore, the 1694-1734 construct corresponded to the sequence of a genotype 1a HCV, whereas the 1616-1862 construct corresponded to a genotype 1b HCV, and the 42 residue construct (1694-1734) had to 2 amino acid sequences difference from the corresponding region of the 1616-1862 construct from genotype 1b HCV, demonstrating that the immunomodulatory activity of this region is retained across different variants of HCV. This suggests that variants or mutants constructs of NS4 may have similar or enhanced immunomodulatory activity to that observed with sequences from genotype 1a and 1b.

NS4 plays an important role in the viral life cycle, acting as a cofactor for the NS3 serine protease (38). Together these proteins are responsible for most of the cleavages occurring in the non-structural region of the polyprotein. NS4 is believed to be either membrane-bound or secreted from infected cells, and does not form part of the virion particle. As well as being involved in viral replication, NS4A and NS4B can inhibit host cell translation and proliferation (39). Furthermore, a recombinant NS3/4A complex has been shown to inhibit cAMP-dependant protein kinase (40).

It was found in the present invention that NS4 inhibited antigen-specific IFN-γ production by PBMC from HCV and normal individuals and IL-12 production by PBMC from normal individuals and induced the production of the immunosuppressive cytokine, IL-10. NS4 induces significant IL-10 production by PBMC from chronically infected patients, and a neutralising IL-10 antibody restored NS4specific IFN-γ production by PBMC from HCV infected donors. Furthermore, purified CD14⁺ monocytes from normal individuals secreted IL-10 in response to NS4, indicating that at least a proportion of the IL-10 observed in vivo during HCV infection, may be derived from cells of the innate immune system. Interestingly CD14⁻ blood monocyte-derived DC did not secrete IL-10, and anti-CD14 blocking antibodies inhibited IL-10 production by monocytes, suggesting that CD14 was directly involved in monocyte IL-10 production. IL-10 was induced by NS4 and not contaminating E.coli products in the recombinant preparation as shown by the demonstration that a) monocyte IL-10 production was significantly reduced following heat-treatment of NS4, b) the NS4 protein was devoid of detectable LPS (less than 4 pg/μg protein) c) NS4 did not stimulate pro-inflammatory cytokines from monocytes, normally induced with low concentrations of LPS and d) NS4 did not induce DC IL-10 production, which was stimulated by LPS.

Induction of IL-10 and inhibition of IL-12 production by cells of the innate immune system has previously been shown to contribute to suppression of cellular immune responses, in particular protective Th1 responses, in a number of chronic or persistent infections, including those caused by HIV, B. pertussis, leishmania and measles virus (16-21, 41). The differentiation of Th1 and Th2 cells from naive T cells is promoted by IL-12 and IL-4 respectively. In contrast, evidence is emerging that molecules that stimulate IL-10 and inhibit IL-12 production by macrophages and DC, including filamentous haemagglutinin from B. pertussis and cholera toxin, may promote the differentiation of Tr1 cells (33). As well as a role in the maintenance of tolerance against self-antigens, Tr cells can be induced against pathogen antigens, especially during chronic infection, where cellular immune responses are suppressed (33). Antigen-specific Tr1 or Th3-type clones have been generated from the respiratory tract of mice infected with B. pertussis (20), and from peripheral blood of humans infected with the filarial parasite Onchocerca volvulus (42). The murine Tr1 clones were shown to suppress IFN-γ production by Th1 cells in vitro and in vivo. HCV core-specific Tr1 clones, as well as Th1 clones, can be isolated from peripheral blood of chronically HCV infected patients (30).

In the present invention it was found that NS4 stimulates IL-10 and inhibits IL-12 production, therefore NS4 has a role in driving Tr1 cells in vivo during HCV infection. The activation of IL-10-secreting Tr cells specific for NS4 and other HCV antigens, including the core protein, provide a positive loop for the amplification of monocyte-derived IL-10 and contribute to suppression of cellular immune responses in chronically HCV infected patients.

DC have previously been shown to have a critical role in directing the induction of T cell subtypes (43). We have found that the regulatory cytokines secreted by monocytes may influence the ability of DC to activate T cells. Supernatants of NS4-stimulated monocytes, that includes IL-10 and possibly other anti-inflammatory cytokines, inhibited maturation and the allo-stimulatory activity of DC, an effect that was partially abrogated by anti-IL-10. Furthermore addition of anti-IL-10 attenuated the inhibitory effect of NS4 on IFN-γ to HCV, in HCV infected patients. Expression of the core protein in DC inhibited their ability to process or present antigen to T cells specific for HCV but not recall antigens (47). In addition, monocyte-derived DCs from chronically infected patients have defective allostimulatory function and reduced expression of CD83 and CD86 (48, 49). We have found that products of NS4-stimulated monocytes inhibited CD83 and CD86 expression on monocyte-derived DC. Therefore cytokines induced by NS4-stimulated monocytes, as well as having a direct affect on IFN-γ production by T cells, may indirectly, by modulating DC activation and altering the cytokine milieu, inhibit the induction of Th1 cells and promote the activation of Tr cells.

Pathogen induction of immunosuppressive cytokines by cells of the innate immune system, amplified through the generation of Tr cells, represent a novel strategy for the pathogen to evade protective cellular immune responses. The combination of elevated IL-10 production, and IL-10-mediated suppression of antigen-specific IFN-γ production in vitro, strongly indicate that IL-10 is a major cause of ineffective anti-pathogen immune responses, particularly adaptive Th1 responses in persistently infected individuals.

Therapies that target immunosuppressive cytokines, specifically IL-10, have valuable therapeutic potential for the treatment of patients chronically infected with HCV.

In addition a Hepatitis C virus (HCV) protein or derivative thereof, in particular HCV NS4 may be exploited as a therapeutic for immune mediated diseases where Th1 responses play a role in inmmunopathology. The HCV protein may be used in the modulation of immune mediated diseases in humans, in particular in those individuals who have not been exposed to the Hepatitis C virus.

HCV protein products may be used in the modulation of inflammatory cytokine production induced by infection or trauma. It may also be used in the treatment of sepsis or acute inflammation induced by infection, trauma or injury. The HCV protein may also be used in the treatment of chronic inflammatory disease, graft rejection or graft versus host disease.

The HCV protein may be used in the treatment of immune mediated diseases involving Th1 responses such as any one or more of Crohns disease, inflammatory bowel disease, multiple sclerosis, type 1 diabetes, rheumatoid arthritis. Since IL-10 and Tr cells can also inhibit inmune responses mediated by Th2 cells, NS4 may be used in the treatment of allergy or asthma.

Agents that induce anti-inflammatory cytokines such as the HCV non-structural protein 4 (NS4) and NS3 will have a direct immunosuppressive effect and will also in the presence of antigen, prime IL-10 secreting antigen-specific Tr cells which will amplify IL-10 production and the immunosuppressive effect.

The Hepatitis C virus (HCV) protein or derivative or mutant or fragment or variant or peptide thereof may be in a form for oral, intranasal, intravenous, subcutaneous, intradermal or intramuscular administration. The HCV protein may be administered in the form of a composition or formulation with a pharmaceutically acceptable carrier and/or in combination with a pharmacologically suitable adjuvant. The composition or formulation may comprise at least one other pharmaceutical product such as an antibiotic.

Materials and Methods

Study subjects. A group of Irish women who were iatrogenically infected with HCV genotype 1b following the administration of contaminated anti-D immunoglobulin in 1977-1978 formed the study cohort (30). Patients who were positive for both anti-HCV antibody and serum HCV-RNA were included. All patients had no apparent history of other types of liver disease. In addition, peripheral blood or buffy coats from healthy volunteers were used as a source of normal peripheral blood mononuclear cells (PBMC). All normal donors tested serologically negative for HCV. Ethical approval was obtained from the St. Vincent's University Hospital and St. James's Hospital Ethics Committees and informed consent was obtained from all patients prior to participation.

Antigens

Recombinant NS4 (rNS4), corresponding to amino acids 1616-1862, of the HCV polyprotein, was purchased from Mikrogen GmbH, Martinsried, Germany, and was free of LPS by analysis with a Limulus Amoebocyte Lysate assay (Biowhittaker). Purification involved a combination of steps, including ion exchange, hydrophobic interaction, chromatographic and preparative SDS-PAA gel. Contaminating LPS was removed during the ion exchange and hydrophobic interaction steps. Recombinant E. coli expressed HCV NS3 was purchased from Mikrogen. rNS4* protein, corresponding to amino acids 1694-1735 of the HCV polyprotein, and HCV E2 were purchased from Austral Biologicals, San Ramon Calif., USA. Influenza virus haemagglutinin C(A) was expressed as a His-tagged protein in E. Coli and purified on a nickel column. E. coli LPS (serotype 127:B8) was purchased from Sigma-Aldrich.

Mikrogen Sequence (NS4):

AA sequence (AA 1616-1862)

Label: None

Genotype 1b
mrgsTLHGPTPLLYRLGAVQNEVTLTHPITKYIMTCMSADLEVVTSTWVL
VGGVLAALAAYCLSTGCVVIVGRIVLSGKPAVIPDREVLYREFDEMEECS
QHLPYIEQGMALAEQFKQKALGLLQTASRQAEVIAPAVQTNWQKLEAFWA
KHMWNFISGIQYLAGLSTLPGNPAIASLMAFTAAVTSPLTTSQTLLFNIL
GGWVAAQLAAPGAATAFVGAGLAGAAIGSVGLGKVLVDILAGYGAGVAGA
LV.

Austral Sequence (NS4):

AA Sequence (AA Ile 1694 to Leu 1735)

Label: N terminal Super Oxide Dismutase

Genotype 1a
IIPDREVLYREFDEMEECSQHLPYIEQGMMLAEQFKQKALGL

Reagents. RPMI-1640 medium (Gibco BRL, NY, USA) supplemented with L-glutamine (2 mM), penicillin (5 mM), steptomycin (5 mM), and 8-10% FCS was used for cell culture. Purified protein derivative of Mycobacterium tuberculosis (PPD) was purchased from Difco Laboratories (Detroit, Mich.). Phorbal mysristate acetate (PMA) was purchased from A. G. Scientific Inc, San Diego, Calif. Recombinant human (rh) GM-CSF was purchased from R&D Systems, UK. rhIL-4 and rhIFN-γ and all antibodies were purchased from BD PharMingen, San Diego, Calif. Phytohemagglutinin (PHA) was purchased from ICN Biomedicals.

NS4-stimulated cytokine production by PBMC. PBMC were isolated from whole blood of HCV antibody positive, polymerase chain reaction (PCR) positive HCV-infected patients, by centrifugation on Ficoll gradients (Histopaque-1077; Sigma Diagnostics, St. Louis, USA). Cells were washed twice and resuspended in RPMI medium with 10% FCS. PBMC (2×10⁶/ml) were stimulated in flat-bottomed 96-well plates with rNS4 (0.4 or 2.0 g/ml) or PHA (20 μg/ml) in RPMI and 10% FCS, in the presence or absence of neutralizing IL-10 (clone JES3-9D7; 10 μg/ml). Cells were incubated for 72 h at 37° C. in a humidified incubator with 5% CO₂. Culture supernatants were removed stored at −20° C. The concentrations of IFN-γ and IL-10 in supernatants were determined by immunoassay using antibody pairs purchased from BD PharMingen as described (30).

Effect of NS4 on proliferation of normal PBMC to recall antigens. PBMC (1×10⁶/ml) from normal donors were stimulated in flat-bottomed 96-well plates with rNS4 (0.4 or 2.0 μg/ml) TT (5 Lf/ml), PPD (500 U/ml) or PMA (0.2 μg/ml) and anti-CD3 (clone HIT3a; 10 μg/ml) in the presence or absence of NS4 (2 μg/ml). Proliferation was measured by ³H Thymidine incorporation on day 3 (PMA, CD3) or day 5 (TT, PPD) of culture.

Purification of adherent cells, T cell enriched and depleted cells and monocytes. Adherent and non-adherent cells were prepared from PBMC by allowing the cells to adhere to plastic for 2 hrs in 6 well plates at 37° C. in humidified 5% CO₂ in air, at a concentration of 2×10⁶/ml. Non-adherent cells were removed by washing several times with warm RPMI medium, and remaining adherent cells were removed using a cell scraper and then washed with RPMI medium. T cell enriched and depleted PBMC were prepared by E resetting. Sheep red blood cells (SRBC) were treated with 2-Aminoethylisothiouronium bromide (AET, Sigma) for 15 mins at 37° C., and washed extensively. PBMC (1×10⁶/ml) were mixed with an equal volume of AET-treated SRBC (1%), incubated at RT for 10 min. The cell suspension was layered onto Ficoll and centrifuged at RT for 10 min at 50 g, and then at 450 g for 30 min at 20° C. The non-rosetting (E⁻) cells were recovered from the interface, washed and resuspended in RPMI medium. The rosette positive (E⁺) cells were recovered from the pellet, washed with RPMI with 8% FCS, and treated with ammonium chloride (NH₄Cl) buffer for 5 mins at RT to lyse erythrocytes. After washing, the cells were resuspended in RPMI at 1×10⁶/ml. CD14⁺ or CD11b⁺ monocytes were isolated from PBMC using positive selection with MACS microbeads (Miltenyi Biotec, GmBH, Bergisch-Gladbach, Germany) and an autoMACS cell sorting instrument. An E⁻ fraction of PBMC was incubated with MACS CD14 or CD11b immunomagnetic beads (Miltenyi Biotec), and allowed to pass through the autoMACS using positive selection. The purity of CD14⁺ and CD11b⁺ monocytes after autoMACS separation were routinely 90-95% as estimated by FACScan analysis using FITC-conjugated CD14 (clone M5E2).

Preparation of monocyte-derived DC. DC were differentiated from MACS-isolated CD14⁺ cells by culture for 7 days in RPMI 1640 and 10% FCS supplemented with granulocyte-macrophage colony-stimulating factor (GM-CSF) (50 ng/ml), and IL-4, (70 ng/ml) in a CO₂ incubator at 37° C. Cultures were fed every 2 days by removing one-half of the supernatant and adding fresh medium and cytokines. FACS analysis revealed that resulting cells were positive for the DC marker CD11c and negative for the human maturation marker CD83, indicating that monocyte-derived DC propagated by this method gave rise to immature DC (iDC).

Induction or inhibition of cytokine production by PBMC, monocytes and DC, PBMC, adherent cells, non-adherent cells, T cells (E⁺), T-cell depleted (E⁻), monocytes (CD11b⁺ or CD14⁺) or monocytes-derived DC (1×10⁶/ml) were stimulated with rNS4 (0.4 and 2.0 μg/ml) in the presence or absence of a neutralizing anti-CD14 mAb (clone M5E2 10 μg/ml) in 24-well plates (NUNC) at 37° C. in humidified 5% CO₂ in air. Supernatants were removed after 24 h and IL-10 concentrations determined by inmmunoassay. The effect of NS4 on IL-12 production was determined by pre-stimulating PBMC (1×10⁶/ml) for 2 h with NS4 (0.4, 2.0 and 10 μg/ml), followed by addition of LPS (1 μg/ml) and IFN-γ (20 ng/ml) and incubation for a further 22 h. Supernatants were removed and concentrations of IL-12 p70 determined by immunoassay.

Modulation of DC surface marker expression. The effect of NS4 on DC maturation was determined directly by adding rNS4 to iDC cultures and indirectly by culturing iDC with products of rNS4-stimulated monocytes. NS4-stimulated monocyte conditioned medium (NS4-MCM) was prepared by stimulating purified monocytes with NS4 (2 μg/ml) and removing the supernatants after 24 h. Monocyte-derived iDC were stimulated with NS4 (2 μg/ml), NS4MCM (10%), LPS (1 μg/ml) and IFN-γ (20 ng/ml) or LPS and IFN-γ and NS4 or NS4MCM. After 24 h cells were recovered, washed, and expression of surface marker on DC was assessed using, PE-conjugated anti-CD86 (clone IT2.2), FITC conjugated anti-CD83 (clone HB15e), and PE-conjugated CD11c (clone B-ly6). All antibodies were purchased from BD PharMingen. Cells incubated with an isotype matched directly conjugated antibody with irrelevant specificity acted as a control. After incubation for 15 mins at RT, cells were washed and immunoflourescence analysis was performed on a FACScan™ (Becton Dickinson) and analyzed using CELLQuest™ software. 10,000 cells were analyzed per sample.

Modulation of DC stimulatory capacity for allo-specific T cells. Supernatants (100 μl) from monocytes cultured in the presence or absence of NS4 (2 μg/ml) were incubated with monocyte-derived DC for 2 h and then washed thoroughly. DC (10³-10⁵/ml were cultured with purified allogeneic T-cells (1×10⁶/ml) in RPMI medium in triplicate wells of 96-well flat-bottomed tissue-culture plates. Supernatants (50 μl) were removed on day 3 of culture for assessment of IFN-γ, IL-5 and IL-10 production, and replaced with fresh medium. Proliferation of T cells was measured by ³H incorporation, over the last 18 h of a 5-day culture.

Results

Defective HCV-specific IFN-γ production by PBMC from chronically infected patients is reversed in the presence of anti-IL-10. The development and maintenance of the chronically infected state during HCV infection has been linked to the presence of Th2 cytokines, especially the anti-inflammatory cytokine IL-10 (9, 10, 27). Synthetic peptides corresponding to the core protein of HCV have been shown to stimulate IFN-γ and IL-10 production by T cells from the chronically infected anti-D cohort of HCV infected patients (30). In this invention the immune response to the HCV NS4 protein and the role of IL-10 in immunosuppression in chronic HCV infection was examined. rNS4 induced IL-10 production by PBMC from all chronic HCV-infected patients examined (FIG. 1). In contrast, IFN-γ production could not be detected in response to NS4 (FIG. 1) or NS3 (not shown) in more than 20 patients examined, but was produced by PBMC in response to PHA. In order to establish whether IL-10 suppressed the NS4specific IFN-γ response in these patients, PBMC were cultured in the presence of a neutralizing IL-10 monoclonal antibody. IFN-γ production to the NS4 protein was significantly increased in the presence of anti-IL-10 (FIG. 1), showing that IL-10 plays an immunosuppressive role in controlling Th1-type responses during HCV infection.

NS4 and NS3 induces IL-10 production in PBMC from normal donors.

Stimulation of PBMC from normal individuals with NS4 induced significant levels of IL-10, without concomitant induction of IFN-γ (FIG. 2A, B), indicating that this protein is capable of inducing IL-10 in a non-specific manner, most likely from cells of the innate immune system. Heat inactivation of the NS4 protein abolished cytokine production (FIG. 2C), suggesting that the IL-10 induction is a receptor-mediated ligation event, and not due to non-protein contaminants in the rNS4 preparation. Furthermore, NS4 failed to induce the production of the pro-inflammatory cytokines, IL-12 (FIG. 5) or TNF-α (not shown) by normal PBMC. PBMC from normal donors were also stimulated with E. coli expressed HCV NS3 and HCV NS4 (0.4 and 2.0 μg/ml) (purchased from Mikrogen), and rNS4* and HCV E2 (purchased from Austral Biologics), influenza virus HA or LPS, at 37° C. in humidified 5% CO₂ in air. Supernatants were removed after 24 hr and IL-10 concentrations determined by immunoassay. Significant levels of IL-10 were detected in PBMC supernatants 24 hours after stimulation with both E. coli-expressed NS4 (Mikrogen), and rNS4* (Austral Biologics), but not with E. coli-expressed influenza virus HA or HCV E2. E. coli expressed NS3 also stimulated IL-10 production by PBMC (FIG. 2D).

NS4 induces IL-10 production in monocytes but not CD14⁻ DCs. To elucidate the cell(s) responsible for NS4-induced IL-10 production, PBMC from normal donors were separated into various cell fractions. Plastic adherent and non-adherent cells in PBMC samples from normal donors were examined and it was found that IL-10 was secreted only by the adherent fraction (FIG. 3). T cell enriched (E⁺) and T cell depleted (E⁻) fractions were examined and were found that IL-10 was secreted only by the non-T cell fraction (FIG. 3). In addition to monocytes/macrophages, immature DC have previously been shown to be a major source of innate IL-10 in response to certain pathogens and play a vital role in the triggering of primary adaptive immune responses to infection (33). Immature DC, expanded from blood monocytes with GM-CSF and IL-4 did not produce IL-10 in response to NS4, but did secrete IL-10 in response to LPS (FIG. 3F). In contrast, CD14⁺ or CD11b⁺ cells, purified from T cell-depleted cells from normal donors, secreted IL-10 in response to NS4 (FIG. 3), indicating that blood monocytes and not blood monocyte-derived DC are the source of HCV-induced innate IL-10.

NS4-induced IL-10 production is mediated by CD14. MACS-purified CD14⁺ monocytes are isolated on the basis of positive selection for CD14. As a result, CD14 antibody-coated magnetic beads occupy many of the CD14 molecules on the purified cell population. The observation that MACS-purified CD14⁺ monocytes stimulated with NS4 produced slightly less IL-10 than un-separated PBMC or T-cell depleted cells (FIG. 3), and that CD14⁺ monocytes but not CD14⁻ DC produce IL-10 in response to NS4, indicating that NS4-induced IL-10 may be dependent on CD14 ligation. PBMC, T-cell depleted fractions of PBMC and purified monocytes, were stimulated with NS4 in the presence or absence of a neutralizing anti-CD14 antibody. In the case of whole PBMC and T-cell depleted PBMC, NS4-induced IL-10 production was significantly inhibited, but not completely abrogated in the presence of anti-CD14 (FIG. 4). However, in the highly purified monocyte preparations, stimulation with NS4 in the presence of anti-CD14 almost completely abolished IL-10 production (FIG. 4), indicating that NS4-induced IL-10 production is mediated by CD14.

NS4 inhibits IL-12 production. IL-12, together with IL-23 and IL-27, play a critical role in the development of cellular immunity against intracellular pathogens, by driving IFN-γ production and regulating the development of Th1 cells (34). PBMC from normal donors were cultured with NS4 for 2 h prior to stimulation with LPS and IFN-γ. Stimulation of PBMC with NS4 only, induced significant IL-10 production, but no detectable IL-12 (FIG. 5). In contrast, high levels of IL-12p70 and IL-10 were detected in the supernatants of PBMC stimulated with LPS and IFN-γ. Pre-incubation of cells with NS4 significantly inhibited IL-12 and augmented IL-10 production in response to LPS and IPN-γ (FIG. 5). Therefore NS4 appears to interfere with IL 12 production. The production of IL-12 in response to Toll-like receptor (TTR) ligands is mediated through the MAP kinase and NFκB signalling. NS4 was found to modulate the NFκB signalling pathway in a macrophage cell line, providing further evidence of its anti-inflammatory and therapeutic potential.

NS4 inhibits T-cell responses to bystander antigens. Addition of NS4 to PBMC significantly reduced the proliferative T-cell response induced by the polyclonal activators, PMA or CD3 and the recall antigen, PPD (FIG. 6). NS4 also inhibited (but not significantly) T-cell proliferation to the recall antigen, TT (FIG. 6B). Therefore NS4 does influence the T-cell response to third party antigens in cells from normal individuals

NS4-stimulated monocytes inhibit DC maturation and stimulation of allo-specific Th1 cells. Since DC, rather than monocytes, play a dominant role in priming naive T cells in vivo and in directing the induction of Th1, Th2 or Tr cells, the influence of the products of NS4-activated monocytes on DC activation and their ability to prime T cells in vitro was examined. CD11b⁺ monocytes isolated from PBMC were stimulated with NS4 and supernatants were removed after 24 h and examined for their effect on maturation and allostimulatory capacity of DCs. Stimulation of blood monocyte-derived iDC with LPS enhanced surface expression of CD83 and CD86 (FIG. 7) In contrast, NS4 did little direct effect on surface expression of these maturation markers on DC. However, supernatants from NS4-stimulated monocytes reduced the percentage of DCs staining positive for CD83 and CD86. Furthermore, supernatants from monocytes stimulated with NS4 inhibited LPS-induced upregulation of CD83 and CD86.

The influence of the products of NS4-stimulated monocytes on the capacity of DC to activate allo-specific T cells was also examined. Monocyte derived DC were incubated with NS4-stimulated monocyte supernatants for 2 h, and subsequently used to stimulate purified allogeneic T-cells. DC treated with control-MCM stimulated proliferation and IFN-γ production by allogeneic T cells. However, proliferation and IFN-γ production by T cells in response to allogeneic DC were significantly reduced, and IL-5 and IL-10 production enhanced, though not significantly, when the DC were pre-incubated with NS4-MCM (FIG. 8). These finding suggest that NS4 indirectly inhibits Th1 and enhances Th2 or Tr1-type responses and that this effect is mediated by the production of soluble factors from monocytes that influence the ability of DCs to activate distinct T cell subtypes.

NS4 has Anti-Inflammatory Activity In Vivo

In order to demonstrate that NS4 had anti-inflammatory activity in vivo, a murine septic shock model was employed Mice were injected with NS4 protein in a PBS solution alone or PBS alone 1 hour prior to administration of LPS (1 μg) and cytokine concentrations in serum were assessed 6 hours later. Injection of NS4 enhanced serum levels of IL-10 and inhibited LPS-induced IFN-γ production. This finding demonstrates that NS4 is active in vivo and is capable of inhibiting inflammatory responses in the murine septic shock model.

The invention is not limited to the embodiments hereinbefore described which may be varied in detail.

### REFERENCES

1. Freeman, A. J., Marinoa, G., French, A., & Lloyd, A. R. (2001) Inununopathogenesis of hepatitis C virus infection. Immunol. Cell. Biol. 79, 515-536.

2. Hoofnagle, J. H. (2002) Course and outcome of hepatitis C. Hepatology 36, S21-29.

3. Rehermann, B. & Chisari, F. V. (2000) Cell mediated inmune response to the hepatitis C virus. Curr. Top. Microbiol. Immunol. 242, 299-325.

4. Gruner, N. H., Gerlach, T. J., Jung, M. C., Diepolder, H. M., Schirren, C. A., Schraut, W. W., Hoffmann, R., Zachoval, R., Santantonio, T., Cucchiarini, M., Cerny, A. & Pape, G. R. (2000). Association of hepatitis C virus-specific CD8⁺ T cells with viral clearance in acute hepatitis C. J. Infect. Dis. 181, 1528-1536.

5. Chang, K. M., Thimme, R., Melpolder, J. J., Oldach, D., Pemberton, J., Moorhead-Loudis, J., McHutchison, J. G., Alter, H. J. & Chisari, F. V. (2001) Differential CD4(+) and CD8(+) T-cell responsiveness in hepatitis C virus infection. Hepatology 33, 267-276.

6. Day, C. L., Lauer, G. M., Robbins, G. K., McGovern, B., Wurcel, A. G., Gandhi, R. T., Chung, R. T., & Walker, B. D. (2002) Broad specificity of virus-specific CD4⁺ T-helper-cell responses in resolved hepatitis C virus infection J. Virol. 76, 12584-12595.

7. Koziel, M. I., Dudley, D., Wong, 3. T., Dienstag, J., Houghton, M., Ralston, R., & Walker, B. D. (1992) Intrahepatic cytotoxic T lymphocytes specific for hepatitis C virus in persons with chronic hepatitis. J. Immunol 149, 3339-3344.

8. Battegay, M., Fikes, J., Di Bisceglie, A. M., Wentworth, P. A., Sette, A., Celis, E., Ching, W. M., Grakoui, A., Rice, C. M., Kurokohchi, K., Berzofsky, J. A., Hoofnagle, J. H., Feinstone, S. M. & Akatsuka, T. (1995) Patients with chronic hepatitis C have circulating cytotoxic T-cells which recognize hepatitis C virus encoded peptides binding to HLA-A2.1 molecules. J. Virol. 69, 2462-2470.

9. Tsai, S.-L., Liaw, Y.-F., Chen, M.-H., Huang, C.-Y. & Kuo, G. C. (1997) Detection of type-2 like T-helper cells in hepatitis C virus infection: implications for hepatitis C virus chronicity. Hepatology 25, 449-458.

10. Gerlach, J. T., Diepolder, H. M., Jung, M. C., Gruner, N. H., Schraut, W. W., Zachoval, R., Hoffmann, R., Schirren, C. A., Santantonio, T. & Pape, G. R. (1999) Recurrence of hepatitis C virus after loss of virus-specific CD4(+) T-cell response in acute hepatitis C. Gastroenterology 117, 933-941.

11. Napoli, J., Bishop, G. A., McGuinness, P. H., Painter, D. M., & McCaughan, G. W. (1996) Progressive liver injury in chronic hepatitis C infection correlates with increased intrahepatic expression of Th1-associated cytokines. Hepatology 24, 759-765.

12. Quiroga, J. A., Martin, J., Navas, S., & Carreno, V. (1998) Induction of interleukin-12 production in chronic hepatitis C virus infection correlates with the hepatocellular damage. J. Infect Dis. 178, 247-251.

13. Sobue, S., Nomura, T., Ishikawa, T., Ito, S., Saso, K., Ohara, H., Joh, T., Itoh, M., & Kakumu, S. (2001) Th1/Th2 cytokine profiles and their relationship to clinical features in patients with chronic hepatitis C virus infection. J. Gastroenterol. 36, 544-551.

14. Hengel, H. & Koszinowski, U. H. (1997) Interference with antigen processing by viruses. Curr. Opin. Immunol. 9:470-476.

15. Vossen, M. T., Westerhout, E. M., Soderberg-Naucler, C., & Wiertz, E. J. (2002) Immunogenetics Viral immune evasion: a masterpiece of evolution. 54, 527-542.

16. Borghi, P., Fantuzzi, L., Varano, B., Gessani, S., Puddu, P., Conti, L., Capobianchi, M. R., Ameglio, F. & Belardelli, F. (1995) Induction of interleukin-10 by human immunodeficiency virus type 1 and its gp120 protein in human monocytes/macrophages. J. Virol. 69, 1284-1287.

17. Taoufik, Y., Lantz, O., Walion, C., Charles, A., Dussaix, E. & Delfraissy, J. F. (1997). Human immunodeficiency virus gp120 inhibits interleukin-12 secretion by human monocytes: an indirect interleukin-10-mediated effect. Blood 89, 2842-2848.

18. Stockl, J., Vetr, H., Majdic, O., Zlabinger, G., Kuechler, E., & Knapp W. (1999) Human major group rhinoviruses downmodulate the accessory function of monocytes by inducing IL-10. J. Clin. Invest. 104, 957-965.

19. Peacocok, J. W. & Bost K. L. (2001). Murine gammaherpes virus-68-induced interleukin increases viral burden, but limits virus-induced splenomegaly and leulcocytosis. Immunol 104, 109-111.

20. McGuirk, P., McCann, C. & Mills, K. H. G. (2002) Pathogen-specific T regulatory 1 cells induced in the respiratory tract by a bacterial molecule that stimulates interleukin 10 production by dendritic cells: a novel strategy for evasion of protective T helper type 1 responses by Bordetella pertussis J. Exp. Med. 195, 221-231.

21. Barnes, P. F., Chatterjee, D., Abrams, J. S., Lu, S., Wang, E., Yamamura, M., Brennan, P. J., & Modlin, R. L. (1992) Cytokine production induced by Mycobacterium tuberculosis lipoarabinomannan. Relationship to chemical structure. J. Immunol. 149, 541-547.

22. Carvalho, E. M., Bacellar, O., Brownell, C., Regis, T., Coffman, R. L. & Reed, S. G. (1994) Restoration of IFN-γ production and lymphocyte proliferation in visceral leishmaniasis. J. Immunol. 152, 5949-5946.

23. Mahanty, S., Ravichandran, M., Raman, U., Jayaramann, K., Kumaraswami, V. & Nutman, T. B. (1997) Regulation of parasite antigen-driven immune responses by interleukin-10 (IL-10) and IL-12 in lymphatic filariasis. Infect. Immun. 65, 1742-1747.

24. King, C. L., Medhat, A., Malhotra, I., Nafeh, M., Helmby, A., Khaudary, J., Ibrahim, S., El-Sherbiny, M., Zaky, S., Stupi, R. J., Brustoski, K., Shehata, A. & Shata, M. T. (1996) Cytokine control of parasite-specific anergy in human urinary schistsosomiasis. IL-10 modulates lymphocyte reactivity. J. Immunol. 156, 4715-4721.

25. Sieling, P. A., Abrams, J. S., Yamamura, M., Salgame, P., Bloom, B. R., Rea, T. H. & Modlin, R. L. (1993) Immunosuppressive roles for IL-10 and IL-4 in human infection. In vitro modulation of T-cell responses in leprosy. J. Immunol 150, 5501-5510.

26. Gong, J. H., Zhang, M., Modlin, R. L., Linsey, P. S., Iyer, D., Lin, Y., & Branes, P. F. (1996) Interleukin-10 downregulates Mycobacterium tuberculosis-induced Th1 responses and CTLA-4 expression. Infect. Immun. 64, 913-918.

27. Woitas, R. P., Lechmann, M., Jung, G., Kaiser, R., Sauerbruch, T. & Spengler, U. (1997) CD30 induction and cytokine profiles in hepatitis C virus core-specific peripheral blood T lymphocytes J. Immunol. 159, 1012-1018.

28. Kakumu, S., Okamura, A., Ishikawa, T., Iwata, K., Yano, M. & Yoshioka, K. (1997) Production of interleukins 10 and 12 by peripheral blood mononuclear cells (PBMC) in chronic hepatitis C virus (HCV) infection. Clin. Exp. Immunol. 108, 138-143.

29. Reiser, M., Marousis, C. G., Nelson; D. R., Lauer, G., Gonzalez-Peralta, R. P., Davis, G. L., & Lau, J. Y. (1997) Serum interleukin 4 and interleukin 10 levels in patients with chronic hepatitis C virus infection. J. Hepatol. 26, 471-478.

30. MacDonald, A. J., Duffy, M., Brady, M. T., McKiernan, S., Hall, W., Hegarty, J., Curry, M. & Mills, K. H. G. (2002) CD4 T helper type 1 and regulatory T cells induced against the same epitopes on the core protein in hepatitis C virus-infected persons. J. Infect. Dis. 185, 720-727.

31. Scheiner, P. A., Florman, S. S., Emre, S., Fishbein, T., Schwartz, M. E., Miller, C. M. & Boros, P. (2001) Recurrence of hepatitis C after liver transplantation is associated with increased systemic IL-10 levels. Mediators Inflamm 10, 37-41.

32. Yee, L. J., Tang, J., Gibson, A. W., Kimberly, R., Van Leeuwen, D. J., & Kaslow, R. A. (2001) Interleukin 10 polymorphisms as predictors of sustained response in antiviral therapy for chronic hepatitis C infection. Hepatology 33, 708-712.

33. McGuirk, P. & Mills K. H. G. (2002) Pathogen-specific regulatory T cells provoke a shift in the Th1/Th2 paradigm in immunity to infectious diseases. Trends Immunol. 23, 450-455.

34. Robinson, D. S. & O'Garra, A. (2002) Further checkpoints in Th1 development. Immunity 16, 755-758.

35. Gale, M., Jr., Blakely, C. M., Kwieciszewski, B., Tan, S. L., Dossett, M., Tang, N. M., Korth, M. J., Polyak, S. J., Gretch, D. R., & Katze, M. G. (1998) Control of PKR protein kinase by hepatitis C virus nonstructural 5A protein: molecular mechanisms of kinase regulation. 18, Mol. Cell. Biol. 5208-5218.

36. Waris, G., Tardif, K. D., & Siddiqui, A. (2002) Endoplasmic reticulum (ER) stress: hepatitis C virus induces an ER-nucleus signal transduction pathway and activates NF-kappaB and STAT-3. Biochem. Pharmacol. 64, 1425-1430.

37. Bode, J. G., Ludwig, S., Ehrhardt, C., Erhardt, A., Albrecht, U., Schaper, F., Heinrich, P. C., & Haussinger, D. (2003) IFN-alpha antagonistic activity of HCV core protein involves induction of suppressor of cytokine signaling-3 FASEB J. [epub ahead of print]

38. De Francesco, R., & Steinkuhler, C. (2000) Structure and function of the hepatitis C virus NS3-NS4A serine proteinase. Curr. Top. Microbiol. Immunol. 242, 149-169.

39. Kato, J., Kato, N., Yoshida, H., Ono-Nita, S. K., Shiratori, Y., & Omata, M. (2002) Hepatitis C virus NS4A and NS4B proteins suppress translation in vivo. J. Med. Virol. 66, 187-199.

40. Aoubala, M., Holt, J., Clegg, R. A., Rowlands, D. J., & Harris, M. (2001) The inhibition of cAMP-dependent protein kinase by full-length hepatitis C virus NS3/4A complex is due to ATP hydrolysis 82, J. Gen. Virol. 1637-1646.

41. Marie, J. C., Kehren, J., Trescol-Biemont, M. C., Evlashev, A., Valentin, H., Walzer, T., Tedone, R., Loveland, B., Nicolas, J. F., Rabourdin-Combe, C. & Horvat, B. (2001) Mechanism of measles virus-induced suppression of inflammatory immune responses. Immunity 14, 69-79.

42. Doetze, A., Satoguina, J., Burchard, G., Rau, T., Löliger, C., Fleisher, B., & Hoerauf, A. (2000) Antigen-specific cellular hyporesponsiveness in a chronic human helminth infection is mediated by Th3/Tr1-type cytokines IL-10 and transforming growth factor-β but not by a Th1 to Th2 shift Int. Immunol. 12, 623-630.

43. Moser, M. & Murphy, K. M. (2000) Dendritic cell regulation of TH1-TH2 development. Nat. Immunol. 1, 199-205.

44. Tabatabai, N. M., Bian, T. H., Rice, C. M., Yoshizawa, Gill, J., & Eckels, D. D. (1999) Functionally distinct T-cell epitopes within the hepatitis C virus non-structural 3 protein. Hum. Immunol. 60, 105-115.

45. Lee, C. H, Choi, Y. H, Yang, S. H. Lee, C. W., Ha, S. J. & Sung, Y. C. (2001) Hepatitis C virus core protein inhibits interleukin 12 and nitric oxide production from activated macrophages. Virology. 279, 271-279.

46. Yao, Z. Q., Nguyen, D. T., Hiotellis, A. I., & Hahn, Y. S. (2001) Hepatitis C virus core protein inhibits human T lymphocyte responses by a complement-dependent regulatory pathway. J. Immunol. 167, 5264-5272.

47. Sarobe, P., Lasarte, J. J., Casares, N., Lopez-Diaz de Cerio, A., Baixeras, E., Labarga, P., Garcia, N., Borras-Cuesta, F., & Prieto, J. (2002) Abnormal priming of CD4(+) T cells by dendritic cells expressing hepatitis C virus core and E1 proteins. J. Virol. 76, 5062-5070.

48. Bain, C., Fatmi, A., Zoulim, F., Zarski, J. P., Trepo, C., & Inchauspe, G. (2001) Impaired allostimulatory function of dendritic cells in chronic hepatitis C infection. Gastroenterology 120, 512-524.

49. Auffermann-Gretzinger. S., Keeffe, E. B., & Levy. S. (2001) Impaired dendritic cell maturation in patients with chronic, but not resolved, hepatitis C infection. Blood. 97, 3171-3176.

1-54. (canceled)

55: A therapeutic composition comprising a Hepatitis C virus (HCV) agent comprising a HCV protein or derivative or mutant or fragment or variant or peptide thereof or product of cells activated by the agent which suppresses inflammatory cykotine production and/or promotes IL-10 production in vitro.

56: The composition as claimed in claim 55 wherein the HCV protein is non-structural protein 4 (NS4) or a derivative or mutant or fragment or variant or peptide thereof.

57: The composition as claimed in claim 55 wherein the HCV protein is non-structural protein 3 (NS3) or a derivative or mutant or fragment or variant or peptide thereof.

58: The composition as claimed in claim 55 wherein the agent or product thereof stimulates IL-10 production by peripheral blood mononuclear cells (PBMC) and/or monocytes.

59: The composition as claimed in claim 55 wherein the agent or product thereof inhibits dendritic cell activation.

60: The composition as claimed in claim 55 wherein the agent or product thereof inhibits the induction or activation of Th1 or Th2 cells.

61: The composition as claimed in claim 55 wherein the agent or product thereof promotes the induction or activation of regulatory T cells.

62: The composition as claimed in claim 55 wherein the agent or product thereof modulate toll-like receptor ligand-induced NFκB activation.

63: The composition as claimed in claim 55 wherein the agent modulates inflammatory cytokine production induced by acute infection or trauma.

64: A therapeutic composition comprising HCV non-structural protein 4 (NS4) or a derivative or mutant or fragment or variant or peptide thereof or product of cells activated thereby.

65: A therapeutic composition comprising HCV non-structural protein 3 (NS3) or a derivative or mutant or fragment or variant or peptide thereof or product of cells activated thereby.

66: A vaccine adjuvant comprising a Hepatitis C virus (HCV) agent comprising an HCV protein or derivative or mutant or fragment or variant or peptide thereof or product of cells activated by the agent.

67: A vaccine adjuvant comprising HCV non-structural protein 4 (NS4) or a derivative or mutant or fragment or variant or peptide thereof or product of cells activated thereby.

68: A vaccine adjuvant comprising HCV non-structural protein 3 (NS3) or a derivative or mutant or fragment or variant or peptide thereof or product of cells activated thereby.

69: A method for the treatment and/or prophylaxis of an inflammatory and/or immune-mediated disorder and/or disorders associated with transplantation comprising the step of administering an agent comprising a Hepatitis C virus (HCV) protein or derivative or mutant or fragment or variant or peptide thereof or product cells activated by the agent.

70: The method as claimed in claim 69 wherein the HCV protein is non-structural protein 4 (NS4) or a derivative or mutant or fragment or variant or peptide thereof.

71: The method as claimed in claim 69 wherein the HCV protein is non-structural protein 3 (NS3) or a derivative or mutant or fragment or variant or peptide thereof.

72: The method as claimed in claim 69 wherein the agent suppresses inflammatory cytokine production.

73: The method as claimed in claim 69 wherein the agent promotes IL-10 production.

74: The method as claimed in claim 69 wherein the agent stimulates IL-10 production by peripheral blood mononuclear cells (PBMC) and/or monocytes.

75: The method as claimed in claim 69 wherein the agent or product thereof inhibits dendritic cell activation.

76: The method as claimed in claim 69 wherein the agent or product thereof inhibits the induction or activation of Th1 or Th2 cells.

77: The method as claimed in claim 69 wherein the agent or product thereof modulates toll-like receptor (TLR) dependant signalling.

78: The method as claimed in claim 69 wherein the agent modulates inflammatory cytokine production induced by infection or trauma.

79: The method as claimed in claim 69 wherein the disorder is a sepsis or acute inflammation induced by infection, trauma or injury.

80: The method as claimed in claim 69 wherein the disorder is a chronic inflammatory disease, graft rejection or graft versus host disease.

81: The method as claimed in claim 69 wherein the disorder is an immune mediated disease involving Th1 responses.

82: The method as claimed in claim 69 wherein the agent is used for the prophylaxis and/or treatment of diseases or conditions involving toll-like receptor (TLR) dependant signalling.

83: The method as claimed in claim 69 wherein the disorder is an immune mediated disease involving inflammatory cytokines, including TNF-α and IL-1.

84: The method as claimed in claim 69 wherein the disorder is any one or more of Crohn's disease, inflammatory bowel disease, multiple sclerosis, type 1 diabetes systemic lupus erythematosus, uveitis, rheumatoid arthritis, allergy or asthma.

85: A method of inhibiting Toll-like receptor (TLR) dependant signalling comprising administration of an effective amount of Hepatitis C virus (HCV) protein or a derivative, mutant, variant, fragment or peptide thereof.

86: A method for the treatment of infectious disease or cancer comprising the step of administering an agent comprising a Hepatitis C virus (HCV) protein or derivative or mutant or fragment or variant or peptide thereof.

87: A method for the treatment of and/or prophylaxis of asthma and/or allergy comprising the step of administering an agent comprising a Hepatitis C virus (HCV) protein or derivative or mutant or fragment or variant or peptide thereof.

88: The method as claimed in claim 69 wherein the agent is in a form for oral, intranasal, intravenous, intradermal, subcutaneous or intramuscular administration.
